(19) United States
(12) Patent Application Publication
(10) Pub. No.: US 2006/0051811 A1
(43) Pub. Date: Mar. 9, 2006

(54) Site-specific labelling of proteins using acridone and quinacridone lifetime dyes

(76) Inventor: Graham Cotton, Lothian (GB)

Correspondence Address: AMERSHAM BIOSCIENCES;PATENT DEPARTMENT, 800 CENTENNIAL AVENUE, PISCATAWAY, NJ, 08855, US

(21) Appl. No.: 10/522,665

(22) PCT Filed: Jul. 28, 2003
(86) PCT No.: PCT/GB03/03190

§ 371 Date: Jan. 27, 2005

Related U.S. Application Data
(63) Continuation-in-part of application No. 10/241,355, filed on Sep. 11, 2002.

(30) Foreign Application Priority Data
Jul. 30, 2002 (GB) 0217562.8
Jul. 30, 2002 (GB) 02 17562.8

(51) Int. Cl. G01N 33/53; C07D 471/02; C07D 219/04; C07K 14/47

(52) U.S. Cl. 435/7.1; 546/49; 546/103; 530/409

(57) ABSTRACT

Disclosed are compounds of formula: 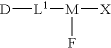 in which D is a fluorescent dye selected from an acridone and a quinacridone dye; F comprises a target bonding group selected from a carboxylic acid thioester group and a 1,2-aminothiol group; M is a group adapted for attaching to F; X is selected from hydrogen or the group:
-L²-B
wherein B is an affinity tag; and L¹ and L² each independently comprise a group containing from 1 to 40 linked atoms selected from carbon atoms which may optionally include one or more groups selected from —NR′—, —O—, —CH═CH—, —CO—NH— and phenylenyl groups, where R′ is selected from hydrogen and C₁-C₄ alkyl. The invention also relates to methods that afford direct attachment of the acridone or quinacridone dye to either the N-terminus or C-terminus of a synthetic or recombinant peptide or protein, and their derivatives, in a site-specific manner, coupled with purification of the resultant labelled molecule.

The present invention relates to reagents and methods for site-specific labelling of proteins with acridone and quinacridone dyes. In particular, the invention relates to new acridone and quinacridone dye derivatives containing thioester activated groups and groups reactive with target molecules containing or derivatised to contain a thioester reactive moiety.

There is an increasing interest in, and demand for, fluorescent labels for use in the labelling and detection of biomolecules. Acridones and quinacridones are highly fluorescent molecules and new acridone and quinacridone dye derivatives having characteristic fluorescence lifetimes have been described for use as labels for target materials. The fluorescence lifetimes of the acridone and quinacridone dyes are generally longer than the lifetimes of other fluorescent labels, as well as naturally occurring fluorescent materials, such as proteins and polynucleotides. The use of fluorescent acridone and quinacridone dyes as fluorescent labels enables easy discrimination from background fluorescence in assays utilising such dyes.

In many applications there is a need to form a permanent link, in the form of a covalent bond, between a fluorescent labelling dye and a target molecule such as a protein. The chemistry of peptide and protein labelling is well documented and a wide range of reagents is now commercially available for the chemical modification of peptides. For a review and examples of protein labelling using fluorescent labelling reagents, see “Non-Radioactive Labelling, a Practical Introduction”, Garman, A. J. Academic Press, 1997; “Handbook of Fluorescent Probes and Research Chemicals”, Haugland, R. P., Molecular Probes Inc., 1992).

Site-specific incorporation of a fluorescent label into a protein or peptide may be of considerable benefit in certain biochemical and biophysical studies, for example fluorescence resonance energy transfer and protein structure and function studies. One method for the site-specific attachment of reporter groups into a target polypeptide utilises the native chemical ligation reaction. According to this procedure, an unprotected peptide fragment containing an N-terminal cysteine residue and a second reporter-labelled peptide fragment containing an α-thioester group are chemoselectively ligated together at physiological pH, irrespective of their primary sequences, to generate an amide bond at the ligation site. For examples, see reviews by Cotton, G. J. and Muir T. W., Chem. Biol., (1999), 6, R247-260; Giriat, I., Muir, T. W. and Perler, F. B., Genetic Engineering, (2001), 23,171-199; Muir, T. W., Syn. Lett., (2001), 6, 733-740.

Tolbert, T. J. and Wong, C-H. (Angew. Chem. Int. Ed., (2002), 41, 2171-2174) describe the preparation of fluorescein and biotin thioester derivatives and the reaction of these with N-terminal cysteine-containing recombinant proteins. Schuler, B. and Pannell, L. K. (Bioconjugate Chemistry, 18 Jul. 2002; published on line) reported the preparation of a benzyl thioester of Cy5™ and subsequent reaction with a synthetic polypeptide containing an N-terminal cysteine residue.

However, there are no reports describing thioester derivatives of fluorescent lifetime reporter molecules such as the acridone and quinacridone dyes, or in which the fluorescence lifetime reporter is additionally linked covalently to an affinity tag. One of the advantages of these reporters is that their characteristic fluorescence lifetime signatures enables target molecules labelled with such dyes to the differentiated from background fluorescence. Additionally, the acridone and quinacridone classes of dyes may be engineered to have a range of fluorescence lifetimes, distinguishable one from the other, thus enabling applications requiring multiplex detection.

The present invention provides reagents and methods that afford direct attachment of a fluorescent acridone or quinacridone dye to either the N-terminus or C-terminus of a synthetic or recombinant peptide or protein, and their derivatives, in a site-specific manner, coupled with purification of the resultant labelled molecule.

According to one aspect of the present invention, there is provided a compound selected from an acridone and a quinacridone dye containing at least one target bonding group selected from a carboxylic acid thioester group or a group suitable for covalent reaction with a thioester, wherein said compound optionally includes an affinity tag covalently bound thereto.

Suitably, the compound is of formula (I): 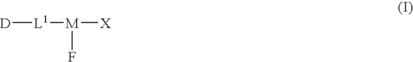
wherein:
D is a fluorescent dye selected from an acridone and a quinacridone dye;
F comprises a target bonding group selected from a carboxylic acid thioester group and a 1,2-aminothiol group;
M is a group adapted for attaching to F;
X is selected from hydrogen or the group:
   -L²-B
   wherein B is an affinity tag; and
L¹ and L² each independently comprise a group containing from 1-40 linked atoms selected from carbon atoms which may optionally include one or more groups selected from —NR′—, —O—, —CH═CH—, —CO—NH— and phenylenyl groups, where R′ is selected from hydrogen and C₁-C₄ alkyl.

Suitably, there are 2 to 30 atoms in each of L¹ and L², preferably, 6 to 20 atoms.

Preferably, L¹ and L² are independently selected from the group:
—{(CHR′)ₚ-Q—(CHR′)ᵣ}ₛ—
where Q is selected from: —CHR′—, —NR′—, —O—, —CH═CH—, —Ar— and —CO—NH—; R′ is hydrogen or C₁-C₄ alkyl, p is 0-5, r is 1-5 and s is 1 or 2.

Particularly preferred Q is selected from: —CHR′—, —O— and —CO—NH—, where R′ is hereinbefore defined.

In a preferred embodiment, group X in the compounds of formula (I) is the group:
—-L²-B
wherein B and L² are hereinbefore defined. In this embodiment, L² is optionally a cleavable linker group and may additionally include group P which may be suitably selected from a chemically-cleavable group, an enzyme-cleavable group, or a photochemically-cleavable group. Suitable chemically cleavable groups include carbamate esters and carboxylate esters, which are both cleaved under basic conditions. Suitable enzyme cleavable groups may be selected from groups such as ester, amide and phospho-diester groups. Such groups are substrates for, and are hydrolysed by hydrolases, such as proteases, esterases and phospho-diesterases. Suitable photocleavable groups P for use in the compound of formula (I) may contain the 4,5-dialkoxy-2-nitrobenzyl alcohol linker (Holmes, C. P., and Jones, D. G., J. Org. Chem., (1995), 60, 2318-2319) or phenacyl linkers (Wang, S., J. Org. Chem., (1976), 41, 3258-3261). These groups undergo efficient photoreaction upon 300 nm illumination, resulting in the rapid cleavage of the dye molecule or dye-labelled protein from the affinity tag.

Suitably, the group M may be any suitable functional group adapted for attaching the target bonding group F. Preferably, M is selected from: 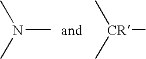
wherein R′ is hereinbefore defined.

Suitable affinity tags may be selected from biotin, desthiobiotin and metal chelating ligands such as his-tag and iminodiacetic acid, nitrilotriacetic acid and the like. Preferred affinity tags may be selected from biotin and desthiobiotin.

In one embodiment of the present invention, the target bonding group F is a carboxylic acid thioester of formula: 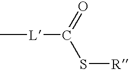
wherein L′ is a bond or is a group containing from 1-30 linked atoms selected from carbon atoms and optionally one or more groups selected from —NH—, —O— and —CO—NH—; and R″ is C₁-C₄ alkyl, C₆-C₁₀ aryl, or C₇-C₁₅ aralkyl, which may be optionally substituted with sulphonate; or is the group —(CH₂)₂—CONH₂. In the case where L′ is a bond, the target bonding group F is attached directly to group M.

In an alternative embodiment, the target bonding group F is a 1,2-aminothiol group of formula: 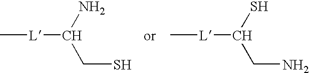
wherein L′ is hereinbefore defined.

Thus, the present invention provides fluorescent labelling reagents comprising an acridone dye or a quinacridone dye, modified by incorporating a target bonding group, and optionally an affinity tag into the molecule. The target bonding group may be selected from a carboxylic acid thioester group or a 1,2-aminothiol group, wherein the thioester group is selectively reactive with a 1,2-aminothiol group on a target molecule, suitably a protein or peptide, or a derivative thereof. In the alternative, the acridone or quinacridone dye may contain a 1,2-aminothiol group for reaction with a thioester group on the target. The incorporation of a reactive thioester or, alternatively, a 1,2-aminothiol functionality into the chemical structure of the reporter groups enables the target molecule to be directly labelled in a convenient one step process. According to the methods of the invention, labelling of peptides and proteins is site-specific, irrespective of the composition of the primary sequence. By generating the target primary sequence with either an N-terminal cysteine or a thioester functionality, site-specific labelling can be achieved directly, by incubating the target with the appropriate derivative of the acridone or quinacridone dye, the α-thioester and 1,2-aminothiol derivatives respectively. The inclusion of an affinity tag in the labelling reagent allows subsequent purification of the fluorescent dye-labelled protein or peptide.

In one embodiment according to the first aspect, the compound is an acridone dye having the formula (II): 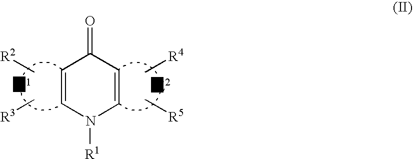
wherein:
groups R² and R³ are attached to the Z¹ ring structure and groups R⁴ and R⁵ are attached to the Z² ring structure;
Z¹ and Z² independently represent the atoms necessary to complete one ring or two fused ring aromatic or heteroaromatic systems, each ring having five or six atoms selected from carbon atoms and optionally no more than two atoms selected from oxygen, nitrogen and sulphur;
at least one of groups R¹, R², R³, R⁴ and R⁵ is a group W having the formula: 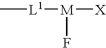
   where F, M, X and L¹ are hereinbefore defined;
when any of said groups R¹, R², R³, R⁴ and R⁵ is not said group W, said remaining groups R², R³, R⁴ and R⁵ are independently selected from hydrogen, halogen, amide, cyano, mono- or di-C₁-C₄ alkyl-substituted amino, carbonyl, carboxyl, C₁-C₆ alkoxy, acrylate, vinyl, styryl, aryl, heteroaryl, C₁-C₂₀ alkyl, aralkyl, sulphonate, sulphonic acid, quaternary ammonium and the group —(CH₂)ₙ—Y and,
when group R¹¹ is not said group W, it is selected from hydrogen, C₁-C₂₀ alkyl, aralkyl and the group —(CH₂)ₙ—Y; and
Y is selected from sulphonate, sulphate, phosphonate, phosphate, quaternary ammonium and carboxyl; and n is an integer from 1 to 6;
provided that at least one of groups R¹, R², R³, R⁴ and R⁵ is a water solubilising group.

In a second embodiment according to the first aspect, the compound is a quinacridone dye having the formula (III): 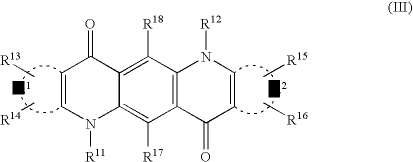
wherein:
groups R¹³ and R¹⁴ are attached to the Z¹ ring structure and groups R¹⁵ and R¹⁶ are attached to the Z² ring structure;
Z¹ and Z² independently represent the atoms necessary to complete one ring or two fused ring aromatic or heteroaromatic systems, each ring having five or six atoms selected from carbon atoms and optionally no more than two atoms selected from oxygen, nitrogen and sulphur;
at least one of groups R¹¹, R¹², R¹³, R¹⁴, R¹⁵, R¹⁶, R¹⁷ and R¹⁸ is a group T having the formula: 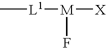
   where F, M, X and L¹ are hereinbefore defined;
when any of said groups R¹³, R¹⁴, R¹⁵, R¹⁶, R¹⁷ and R¹⁸ is not said group T, said remaining groups R¹³, R¹⁴, R¹⁵, R¹⁶, R¹⁷ and R¹⁵ are independently selected from hydrogen, halogen, amide, cyano, mono- or di-C¹-C₄ alkyl-substituted amino, carbonyl, carboxyl, C₁-C₆ alkoxy, acrylate, vinyl, styryl, aryl, heteroaryl, C₁-C₂₀ alkyl, aralkyl, sulphonate, sulphonic acid, quaternary ammonium and the group —(CH₂)ₙ—Y; and,
when either of groups R¹¹ and R¹² is not said group T, it is selected from hydrogen, C₁-C₂₀ alkyl, aralkyl and the group —(CH₂)ₙ—Y;
Y is selected from sulphonate, sulphate, phosphonate, phosphate, quaternary ammonium and carboxyl; and n is an integer from 1 to 6;
provided that at least one of groups R¹¹, R¹², R¹³, R¹⁴, R¹⁵, R¹, R¹⁷ and R¹⁸ is a water solubilising group.

Suitably, in the compounds according to formula (II) and (III), Z¹ and Z² may be selected independently from the group consisting of phenyl, pyridinyl, naphthyl, anthranyl, indenyl, fluorenyl, quinolinyl, indolyl, benzothiophenyl, benzofuranyl and benzimidazolyl moieties. Additional one, or two fused ring systems will be readily apparent to the skilled person. Preferably, Z¹ and Z² are selected from the group consisting of phenyl, pyridinyl, naphthyl, quinolinyl and indolyl moieties. Particularly preferred Z¹ and Z² are phenyl and naphthyl moieties.

Suitably, at least one of the R groups of the dyes of formula (II) and (III) is a water solubilising group for conferring a hydrophilic characteristic to the compound. Solubilising groups, for example, sulphonate, sulphonic acid and quaternary ammonium, may be attached directly to the aromatic ring structures Z¹ and/or Z² of the compound of formula (I) and (II). Alternatively, solubilising groups may be attached by means of a C₁ to C₆ alkyl linker chain to said aromatic ring structures and may be selected from the group —(CH₂)ₙ—Y where Y is selected from sulphonate, sulphate, phosphonate, phosphate, quaternary ammonium and carboxyl; and n is an integer from 1 to 6. Alternative solubilising groups may be carbohydrate residues, for example, monosaccharides, or polyethylene glycol derivatives. Examples of water solubilising constituents include C₁-C₆ alkyl sulphonates, such as —(CH₂)₃—SO₃⁻ and —(CH₂)₄—SO₃⁻. However, one or more sulphonate or sulphonic acid groups attached directly to the aromatic ring structures of a dye of formula (II) or (III) are particularly preferred. Water solubility may be advantageous when labelling proteins.

In the embodiments according to the first aspect:
i) Aryl is an aromatic substituent containing one or two fused aromatic rings containing 6 to 10 carbon atoms, for example phenyl or naphthyl, the aryl being optionally and independently substituted by one or more substituents, for example halogen, straight or branched chain alkyl groups containing 1 to 10 carbon atoms, aralkyl and alkoxy for example methoxy, ethoxy, propoxy and n-butoxy;
ii) Heteroaryl is a mono- or bicyclic 5 to 10 membered aromatic ring system containing at least one and no more than 3 heteroatoms which may be selected from N, O, and S and is optionally and independently substituted by one or more substituents, for example halogen, straight or branched chain alkyl groups containing 1 to 10 carbon atoms, aralkyl and alkoxy for example methoxy, ethoxy, propoxy and n-butoxy;
iii) Aralkyl is a C₁-C₆ alkyl group substituted by an aryl or heteroaryl group;
iv) Halogen and halo groups are selected from fluorine, chlorine, bromine and iodine.

By virtue of the target bonding group F, the compounds according to the present invention are useful for covalently labelling target biological materials in a site-specific manner for applications in biological detection systems. Suitable target materials include proteins, post-translationally modified proteins, peptides, antibodies, antigens, and protein-nucleic acids (PNAs). The reporter moiety may also be conjugated to species which can direct the path of the reporter within or aid entry to or exit from cells (live or dead); such as for example, long alkyl residues to allow permeation of lipophilic membranes, or intercalating species to localise a reporter in a nucleus or other cellular enclave containing double-stranded DNA.

In a second aspect, there is provided a method for labelling a protein of interest wherein said protein contains or is derivatised to contain an N-terminal cysteine, the method comprising:
i) adding to a liquid containing said protein a compound of formula (I): 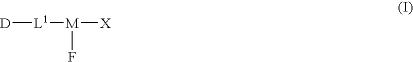
   wherein:
D is a fluorescent dye selected from an acridone and a quinacridone dye;
F comprises a target bonding group selected from a carboxylic acid thioester group and a 1,2-aminothiol group;
M is a group adapted for attaching to F;
X is selected from hydrogen or the group:
   -L²-B
   wherein B is an affinity tag; and
L¹ and L² each independently comprise a group containing from 1-40 linked atoms selected from carbon atoms which may optionally include one or more groups selected from —NR′—, —O—, —CH═CH—, —CO—NH— and phenylenyl groups, where R′ is selected from hydrogen and C₁-C₄ alkyl; and
ii) incubating said compound with said protein under conditions suitable for labelling said protein.

Suitably, there are 2 to 30 atoms in each of L¹ and L², preferably, 6 to 20 atoms.

Preferably, L¹ and L² are independently selected from the group:
—{(CHR′)ₚ-Q—(CHR′)ᵣ}ₛ—
where Q is selected from: —CHR′—, —NR′—, —O—, —CH═CH—, —Ar— and —CO—NH—; R′ is hydrogen or C₁-C₄ alkyl, p is 0-5, r is 1-5 and s is 1 or 2.

Particularly preferred Q is selected from: —CHR′—, —O— and —CO—NH—, where R′ is hereinbefore defined.

Preferably, M is selected from: 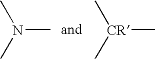
wherein R′ is hereinbefore defined.

Covalent labelling using compounds of the present invention may be accomplished with a target having at least one carboxylic acid thioester group or 1,2-aminothiol group as hereinbefore defined. The target may be incubated with an amount of a compound of the present invention having at least one group F as hereinbefore defined that can covalently bind with the complementary group of the target material. The target material and the compound of the present invention are incubated under conditions and for a period of time sufficient to permit the target material to covalently bond to the compound of the present invention. Thus, for example, the thioester group F may be reacted and form a covalent bond with any of the above target materials that contains, or has been derivatised to contain, a 1,2-amino thiol group. These methods and the products resulting from them, for example, reporter-labelled biomolecules are envisaged as further aspects of the invention.

Suitably, the protein of interest may be selected from the group consisting of antibody, antigen, protein, peptide, microbial materials, cells and cell membranes.

In one embodiment according to the second aspect, there is provided a method of separating and/or purifying the dye-labelled protein of interest by affinity chromatography. In this embodiment, compounds according to the invention include the group:
-L²-B
wherein B and L² are hereinbefore defined. The method utilises the affinity of the affinity tag B covalently attached to the dye, for an immobilised ligand (or specific binding partner) attached to a support material. Affinity chromatography provides a quick and convenient method to enable the separation of labelled and unlabelled protein molecules under physiological conditions. Proteins labelled with an affinity tag can be selectively bound to an affinity column and any unreacted protein removed by washing the column. Suitable specific binding moieties include avidin or streptavidin (for a biotin tag); immobilised metal ions, for example Cu(II), Ni(II), Fe(II) and Fe(III) (for His-tag or iminodiacetic acid). Methods for affinity purification of proteins will be well known to the skilled person, see for example Ostrove, S., Methods in Enzymology, (1990), Vol 182, page 357.

In a typical labelling procedure, a target peptide or protein containing an N-terminal cysteine residue is agitated with an excess of an acridone or quinacridone dye thioester derivative, for example, 9-oxo-10-{6-oxo-6-[2-sulfoethyl)thio]hexyl}-9,10-dihydroacridine-2-sulphonic acid (Ace-MESNA) in buffer (typically 200 mM NaCl, 200 mM sodium phosphate) at ˜pH 7.3-7.4 containing ˜1.5% MESNA. The concentration of the target polypeptide in the labelling reaction is generally between 100 μM to 10 mM, whilst the Ace-MESNA is generally present in excess, for example 1.5 to 3-fold molar excess. When the target polypeptide concentration is relatively low, the concentration of Ace-MESNA is usually maintained at or above 1 mM. Generally, for labelling small peptides a solution of Ace-MESNA and MESNA cofactor is directly added to the lyophilised target.

Typically, for site specific labelling of proteins and large polypeptides using the reagents of the present invention, the target is first transferred into an appropriate buffer, which is known not to effect the labelling reaction. An equal volume of a solution of Ace-MESNA and MESNA thiol co-factor in ligation buffer is then added to the protein to give the desired final concentration of the reactants. The reaction mixture is agitated overnight at room temperature. The reaction time may be lowered to less than one hour for high reactant concentrations and, if the stability of the target polypeptide is an issue, the labelling reaction can be performed efficiently at 4° C. On completion of the labelling reaction, dithiothreitol (DTT) is added to a final concentration of ˜50-250 mM and the desired material isolated by a chromatographic procedure.

Various different denaturants, organic solvents and detergents may be added to the reaction buffer when performing native chemical ligation and expressed protein ligation reactions, to aid the ligation of the peptide fragments and/or stabilise the reactants or products. Such reagents may be utilised in the labelling reaction to increase product yield if necessary. Examples include, but are not limited to guanidinium chloride, urea, dimethylformamide, dimethylsulfoxide, acetonitrile, triton X-100, octyl glucoside, 1,6-hexanediol and glycerol.

The ligation reaction using a derivatised acridone or quinacridone dye according to the present invention may be optimally performed at between pH 7.0 and pH 8.0 and at temperatures varying between 4° C. and 37° C. It is envisaged that such a range of conditions are compatible to the site-specific labelling reaction described herein.

The advantage of the present method is that it enables the introduction of an extrinsic label into a proteinacious substrate in a regioselective and specific manner, thus minimising any detrimental effects that labelling may have on the biological function of the protein. The importance of controlling stoichiometry of labelling is important where dye overload may interfere with biological activity. In addition, if this controlled labelling stoichiometry is directed towards a single terminal site, rather than towards an internal site, this may have the benefit of further maintaining the biological viability of the labelled species.

The invention is further illustrated by reference to the following examples.

### EXPERIMENTAL

### 1. 9-Oxo-10-{6-oxo-6-[2-sulfoethyl)thio]hexyl}-9,10-dihydroacridine-2-sulphonic acid (Ace-MESNA)

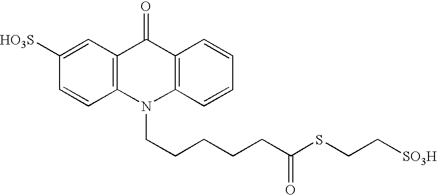

To a stirred solution of 9-oxo-10-{6-carboxyhexyl}-9,10-dihydroacridine-2-sulphonic acid (38.9 mg, 0.1 mmol) and 2-mercaptoethylsulphonic acid, sodium salt (MESNA) (25.2 mg, 0.153 mmol) in anhydrous dimethylformamide (3 ml) at ambient temperature was added a solution of dimethylaminopyridine (13.4 mg, 0.11 mmol) in anhydrous dimethylformamide (1.25 ml) followed by a solution of 1-hyroxybenzotriazole (17.9 mg, 0.132 mmol) in anhydrous dimethylformamide (0.5 ml). To this mixture was added as a solid, dried 4A molecular sieves (˜1 g, <5 micron, activated, powder). The mixture was allowed to stir under a dry nitrogen atmosphere for 30 minutes, and this was followed by addition of N,N′-diisopropylcarbodiimide (126 mg, 155 ul, 1 mmol). The mixture was stirred under a dry nitrogen atmosphere for 15 hours. Thin layer chromatography analysis (reverse phase C18 plates, eluent water/acetonitrile (70:30, containing 0.1% TFA) indicated a major UV visible component (rf 0.43) with no trace of starting material (reference rf 0.34). Work up was by filtration through a glass sinter funnel (porosity 3), washing with dimethylformamide (10 ml), pouring onto cold diethylether (30 ml), and centrifuging (5-10° C., 100 rpm, 10 minutes). After trituration with ethylacetate (2×20 ml) and dissolving the dispersed solid into water (25 ml), HPLC purification was carried out (Phenomenex Jupiter C18 column, 0-60% gradient elution of water/acetonitrile (containing 0.1% TFA) over 40 minutes. The product was obtained as a yellow solid (28.8 mg, 56%), m/z (Maldi) 514, NMR δ_{H} (300 MHz, D₂0) 1.45 (2H, m), 1.72 (4H, m), 2.54 (2H, t, CH₂—CO—S), 3.19 (4H, m, SCH₂CH₂), 4.24 (2H, t, NCH₂), 7.23-8.15 (6H, m, aromatic H) and 8.53 (1H, s, 1-CH).

### 2. Synthesis of Ace-Cys-Gly-Leu-Asp-Lys-Arg-Gly-Cys-Gly-NH₂

### 2.1H-Cys(Trt)-Gly-Leu-Asp(OtBu)-Arg(Pmc)-Lys(Boc)Gly-Cys(Trt)Gly-link amide resin

H-Cys(Trt)-Gly-Leu-Asp(OtBu)-Arg(Pmc)-Lys(Boc)Gly-Cys(Trt)Gly-rink amide resin was synthesised using a commercially available Applied Biosystems Model 433A automated peptide synthesiser using FastMoc™ chemistry, following the instrument manufacturer's recommended procedures throughout. The peptide was synthesised on a 0.25 millimolar scale employing O-(benzotriazol-1-yl) 1,1,3,3-tetramethyluronium hexafluorophosphate (HBTU) as the activating agent.

### 2.2 Cys-Gly-Leu-Asp-Lys-Arg-Gly-Cys-Gly-NH₂

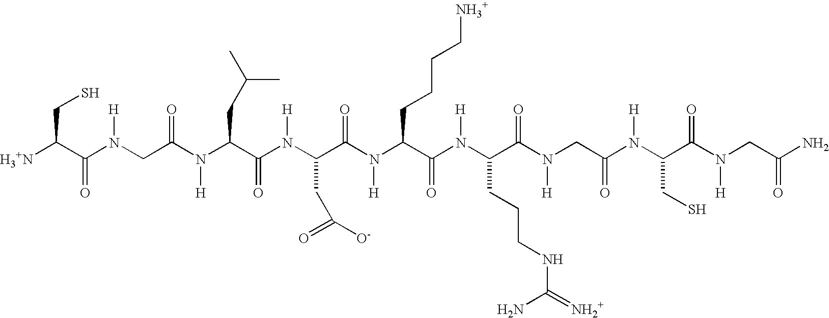

H-Cys(Trt)-Gly-Leu-Asp(OtBu)-Lys(Boc)-Arg(Pmc)-Gly-Cys(Trt)Gly-rink amide resin (100 mg, theoretical loading 0.36 mmol/g) was deprotected and cleaved from solid phase in 95% trifluoroacetic acid (TFA)/2.5% tri-isopropylsilance (TIS)/2.5% water (3 mls) at room temperature for 2 hours. The crude product was precipitated into a 10 fold excess of cold diethyl ether, centrifuged at 2500 rpm for 5 minutes and the ether decanted off. The crude peptide was washed twice more with ether and was purified by reverse phase-high performance liquid chromatography (RP-HPLC) [Phenomenex Jupiter C18 column, eluent A: 0.1% TFA/water, eluent B: 0.1% TFA/acetonitrile, gradient: 0-73% B over 30 mins @1 ml/min, detection at 214 nm]. The product was isolated and lyophilised to afford a colourless fluffy solid (21 mg by weight, 60%). Mono-isotopic mass (as carboxylate): 906.09. Found mass (LC-MS): MH+ @ 907.3; M+ Na @ 929.6; >95% pure as judged by RP-HPLC @ 214 nm (Phenomenex Jupiter C18 column, eluent A: 0.1% TFA/water, eluent B: 0.1% TFA/acetontrile, 5-50% B over 25 mins @ 1 ml/min, UV detection at 650 nm).

### 2.3 Ace-Cys-Gly-Leu-Asp-Lys-Arg-Gly-Cys-Gly-NH₂

To solid H-Cys-Gly-Leu-Asp-Lys-Arg-Gly-Cys-Gly-NH₂ (3.2 mg by weight, 0.0035 mmol) was added a solution of Ace-MESNA (2.5 mg, 0.0049 mmol, 1.4 equivalents) in 200 mM phosphate/200 mM NaCl buffer pH 7.2 containing 1.5% 2-mercaptoethanesulphonic acid, sodium salt (500111). The reaction mixture was stirred for 30 minutes at room temperature in darkness. During incubation, a precipitate formed, which re-dissolved on manual agitation.

A 50 μl aliquot of the reaction mix was removed and to this was added 200 mM phosphate buffer/200 mM NaCl pH 7.2 containing 250 mM (final) dithiothreitol (DTT) (100 μl). The crude reaction mixture was then analysed by RP-HPLC [250×4.6 mm Phenomenex Jupiter C4 column, eluent A: 0.1% TFA/water, eluent B: 0.1% TFA/acetonitrile, gradient; 0-50% B over 35 mins at 1 ml/min, detection at 220 nm and 400 nm]. HPLC indicated over 90% consumption of dye (rt. ˜14.7 min), with major new 400 nm-visible peaks at rt. 18.91, 21.60 and 23.42 mins. The remainder of the reaction mix was then worked up by addition of 200 mM phosphate buffer/200 mM NaCl pH 7.2 containing 250 mM (final) dithiothreitol (DTT) (200 μl). The crude reaction mixture was then subjected to 3 semi-preparative RP-HPLC runs [250×10 mm Phenomenex Jupiter C18 column, eluent A: 0.1% TFA/water, eluent B: 0.1% TFA/acetonitrile, gradient; 0-50% B over 40 mins at 5 ml/min, detection at 220 nm and 400 nm]. The second of four 400 nm-visible peaks (rt. ˜26.5 mins) was identified as the desired product (MALDI-MS, mono-isotopic mass C₅₃H₇₈N₁₄O₁₇S₃ requires 1277, found MH+ 1278). The product from the third HPLC run was isolated and lyophilised as a pale yellow solid (1.4 mg by weight; ˜54.5% yield; 97% pure as judged by RP-HPLC at 400 nm wavelength. Additional identification was carried out: (LC-MS ES+ single component gives MH+ @ 1278).

### 2.4 Characterisation of Labelled Peptide

### 2.4.1 Enzyme Digestion of Ace-Cys-Gly-Leu-Asp-Lys-Arg-Gly-Cys-Gly-NH₂

To a solution of Ace-Cys-Gly-Leu-Asp-Lys-Arg-Gly-Cys-Gly-NH₂ (˜750 μg from the first two preparative HPLC runs from Experimental Section 4.3) in 50 mM TRIS buffer pH 7.9 (500 μl) containing 0.005% Tween was added Endo Asp-N (2 μg) in 50 mM TRIS buffer pH 7.9 (100 μl). The reaction mixture was stirred overnight at room temperature in the dark under an atmosphere of nitrogen. The reaction mixture was treated with 50 mM TRIS buffer pH 7.9 (200 μl) containing 250 mM (final) dithiothreitol for 30 minutes. The crude reaction mixture was then analysed by RP-HPLC [250×4.6 mm Phenomenex Jupiter C4 column, eluent A: 0.1% TFA/water, eluent B: 0.1% TFA/acetonitrile, gradient; 0-50% B over 35 mins at 1 ml/min, detection at 220 nm and 400 nm]. HPLC indicated virtual complete enzyme digestion, with a major new 400 nm-visible peak at rt. 24.71 min and trace of starting substrate at rt. 23.09 mins. The crude reaction mixture was then subjected to semi-preparative RP-HPLC [250×1 0 mm Phenomenex Jupiter C18 column, eluent A: 0.1% TFA/water, eluent B: 0.1% TFA/acetonitrile, gradient; 0-50% B over 35 mins at 5 ml/min, detection at 220 nm and 400 nm]. The main 400 nm-visible peak (rt. ˜31.18 mins) was identified as the desired product (97% by peak area) (MALDI-MS, Ace-Cys-Gly-Leu-OH, mono-isotopic mass C₃₀H₃₇N₄O₉S₂ requires 661.78. Found M+H @ 663). In addition, the crude reaction mixture was analysed (LC− MS ES+, diode array detection). Required cleavage product A (Ace-Cys-Gly-Leu) requires 662.79, found MH+ 663. Required cleavage product B (Asp-Lys-Arg-Gly-Cys-Gly-NH₂) requires 634, found MH+ 634. There was no evidence for compound peaks corresponding to non-specific internal Cys residue labelling. The non-cleaved starting material (Ace-Cys-Gly-Leu-Asp-Lys-Arg-Gly-Cys-Gly-NH₂) was also observed as MH₂²⁺ 639.6 (MH+ @1278.2), indicating an endoAsp N enzyme cleavage efficiency of 91.8%.

### 2.4.2 MS Sequence Analysis of Ace-Cys-Gly-Leu-Asp-Lys-Arg-Gly-Cys-Gly-NH₂

Sequence analysis (MS/MS ion sequence β and γ directions) shows consistency with structure with the terminal Ace-Cys residue observed @ M+ 475.06. No evidence of internal Cys labelling was observed in the sequence.

1. A compound having the formula (I): 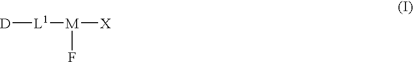
wherein:
D is a fluorescent dye selected from an acridone and a quinacridone dye;
F comprises a target bonding group selected from a carboxylic acid thioester group and a 1,2-aminothiol group;
M is a group adapted for attaching to F;
X is selected from hydrogen or the group;
-L²-B
wherein B is an affinity tag; and
L¹ and L² each independently include a group containing from 1-40 linked atoms selected from carbon atoms which may optionally include one or more groups selected from —NR′—, —O—, —CH═CH—, —CO—NH— and phenylenyl groups, where R′ is selected from hydrogen and C₁-C₄ alkyl.

2. The compound of claim 1, wherein X is a group:
-L²-B.

3. The compound of claim 1, wherein each of L¹ and L² contains from 2 to 30 atoms.

4. The compound of claim 1, wherein L¹ and L² are independently selected from the group:
—{(CHR′)ₚ-Q—(CHR′)ᵣ}ₛ—
where Q is selected from the group consisting of: —CHR′—, —NR′—, —O—, —CH═CH—, —Ar— and —CO—NH—; R′ is hydrogen or C₁-C₄ alkyl, p is 0-5, r is 1-5 and s is 1 or 2.

5. The compound of claim 4, wherein Q is selected from the group consisting of —CHR′—, —O— and —CO—NH—, where R′ is hereinbefore defined.

6. The compound of claim 1, wherein said affinity tag is selected from the group consisting of biotin and desthiobiotin.

7. The compound of claim 1, wherein said affinity tag is selected from the group consisting of his-tag, iminodiacetic acid and nitrilotriacetic acid.

8. The compound of claim 1, wherein the target bonding group F is a carboxylic acid thioester of formula: 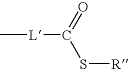
wherein L′ is a bond or is a group containing from 1-30 linked atoms selected from the group consisting of carbon atoms and carbon atoms including one or more groups selected from the group consisting of —NH—, —O— and —CO—NH—; and R″ is C₁-C₄ alkyl, C₆-C₁₀ aryl, or C₇-C₁₅ aralkyl, which may be optionally substituted with sulphonate; or is the group —(CH₂)₂—CONH₂.

9. The compound of claim 1, wherein the target bonding group F is a 1,2-aminothiol group of formula: 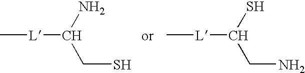
wherein L′ is a bond or is a group containing from 1-30 linked atoms selected from the group consisting of carbon atoms and carbon atoms including one or more groups selected from the group consisting of —NH—, —O— and —CO—NH—.

10. The compound of claim 1, wherein the compound is an acridone dye having the formula (II): 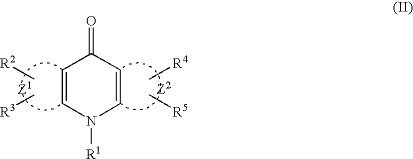
wherein:
groups R² and R³ are attached to the Z¹ ring structure and groups R⁴ and R⁵ are attached to the Z² ring structure;
Z¹ and Z² independently represent the atoms necessary to complete one ring or two fused ring aromatic or heteroaromatic systems, each ring having five or six atoms selected from carbon atoms and optionally no more than two atoms selected from oxygen, nitrogen and sulphur;
at least one of groups R¹, R², R³, R⁴ and R⁵ is a group W having the formula: 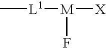
when any of said groups R¹, R², R³, R⁴ and R⁵ is not said group W, said remaining groups R², R³, R⁴ and R⁵ are independently selected from the group consisting of hydrogen, halogen, amide, cyano, mono- or di-C₁-C₄ alkyl-substituted amino, carbonyl, carboxyl, C₁-C₆ alkoxy, acrylate, vinyl, styryl, aryl, heteroaryl, C₁-C₂₀ alkyl, aralkyl, sulphonate, sulphonic acid, quaternary ammonium and the group —(CH₂)ₙ—Y; and
when group R¹ is not said group W, it is selected from the group consisting of hydrogen, C₁-C₂₀ alkyl, aralkyl and the group —(CH₂)ₙ—Y; and
Y is selected from the group consisting of sulphonate, sulphate, phosphonate, phosphate, quaternary ammonium and carboxyl; and n is an integer from 1 to 6;
provided that at least one of groups R¹, R², R³, R⁴ and R⁵ is a water solubilising group.

11. The compound of claim 1, wherein the compound is a quinacridone dye having the formula (III): 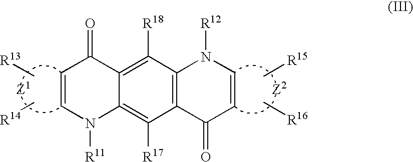
wherein:
groups R¹³ and R¹⁴ are attached to the Z¹ ring structure and groups R¹⁵ and R¹⁶ are attached to the Z² ring structure;
Z¹ and Z² independently represent the atoms necessary to complete one ring or two fused ring aromatic or heteroaromatic systems, each ring having five or six atoms selected from carbon atoms and optionally no more than two atoms selected from oxygen, nitrogen and sulphur;
at least one of groups R¹¹, R¹², R¹³, R¹⁴, R¹⁵, R¹⁶, R¹⁷ and R¹⁸ is a group T having the formula: 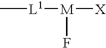
when any of said groups R¹³, R¹⁴, R¹⁵, R¹⁶, R¹⁷ and R¹⁸ is not said group T, said remaining groups R¹³, R¹⁴, R¹⁵, R¹⁵, R¹⁶ and R¹⁸ are independently selected from the group consisting of hydrogen, halogen, amide, cyano, mono- or di-C₁-C₄ alkyl-substituted amino, carbonyl, carboxyl, C₁-C₆ alkoxy, acrylate, vinyl, styryl, aryl, heteroaryl, C₁-C₂₀ alkyl, aralkyl, sulphonate, sulphonic acid, quaternary ammonium and the group —(CH₂)ₙ—Y; and
when either of groups R¹¹ and R¹² is not said group T, it is selected from the group consisting of hydrogen, C₁-C₂₀ alkyl, aralkyl and the group —(CH₂)ₙ—Y;
Y is selected from the group consisting of sulphonate, sulphate, phosphonate, phosphate, quaternary ammonium and carboxyl; and n is an integer from 1 to 6;
provided that at least one of groups R¹¹, R¹², R¹³, R¹⁴, R¹⁵, R¹⁶, R¹⁷ and R¹⁸ is a water solubilising group.

12. The compound of claim 10, wherein Z¹ and Z² are selected independently from the group consisting of phenyl, pyridinyl, naphthyl, quinolinyl and indolyl moieties.

13. The compound of claim 10, wherein Z¹ and Z² are selected from phenyl and naphthyl moieties.

14. A method for labelling a protein of interest wherein said protein contains or is derivatised to contain an N-terminal cysteine, the method comprising:
i) adding to a liquid containing said protein a compound of formula (I): 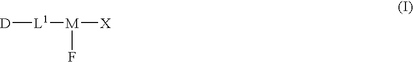
wherein:
D is a fluorescent dye selected from an acridone and a quinacridone dye;
F comprises a target bonding group selected from a carboxylic acid thioester group and a 1,2-aminothiol group;
M is a group adapted for attaching to F;
X is selected from hydrogen or the group:
-L²-B
where B is an affinity tag; and
L¹ and L² each independently include a group containing from 1-40 linked atoms selected from carbon atoms and carbon atoms which may include one or more groups selected from the group consisting of —NR′—, —O—, —CH═CH—, —CO—NH— and phenylenyl groups, where R′ is selected from hydrogen and C₁-C₄ alkyl; and
ii) incubating said compound with said protein under conditions suitable for labelling said protein.

15. The method of claim 14, wherein each of L¹ and L² contains from 2 to 30 atoms.

16. The method of claim 14, wherein L¹ and L² are independently selected from the group:
—{(CHR′)ₚ-Q—(CHR′)ᵣ}ₛ—
where Q is selected from the group consisting of: —CHR′—, —NR′—, —O—, —CH═CH—, —Ar— and —CO—NH—; R′ is hydrogen or C₁-C₄ alkyl, p is 0-5, r is 1-5 and s is 1 or 2.

17. The method of claim 16, wherein Q is selected from the group consisting of —CHR′—, —O— and —CO—NH—, where R′ is hereinbefore defined.

18. The method of claim 14 wherein X is the group:
-L²-B
said method further comprising separating and/or purifying the dye-labelled protein of interest by affinity chromatography.

19. The method of claim 14, wherein said protein of interest is selected from the group consisting of antibodies, antigens, proteins, peptides, microbial materials, cells and cell membranes.

20. The compound of claim 11, wherein Z¹ and Z² are selected independently from the group consisting of phenyl, pyridinyl, naphthyl, quinolinyl and indolyl moieties.

21. The compound of claim 11, wherein Z¹ and Z² are selected from phenyl and naphthyl moieties.
